(19) United States
(12) Patent Application Publication
(10) Pub. No.: US 2025/0306683 A1
(43) Pub. Date: Oct. 2, 2025

(54) A HAPTIC SYSTEM

(71) Applicant: TECHNISCHE UNIVERSITEIT EINDHOVEN, Eindhoven (NL)

(72) Inventors: Danqing LIU, Eindhoven (NL); Dirk Jan BROER, Eindhoven (NL); Mert Orhan ASTAM, Eindhoven (NL); Duygu Sezen POLAT, Eindhoven (NL); Tom BRUINING, Eindhoven (NL)

(21) Appl. No.: 18/864,779

(22) PCT Filed: May 11, 2023
(86) PCT No.: PCT/NL2023/050260

§ 371 Date: Nov. 11, 2024

(30) Foreign Application Priority Data
May 12, 2022 (NL) 2031845

(51) Int. Cl. G06F 3/01; A61B 34/00; B60K 35/25; C09K 19/38

(52) U.S. Cl. CPC G06F 3/016; A61B 34/76; B60K 35/25; C09K 19/3842; B60K 2360/782; C09K 2323/061

(57) ABSTRACT

The present invention relates to a haptic system. The present invention also relates to a method for manufacturing a shape memory liquid crystal network. Furthermore, the present invention relates to electronic apparatuses with user input and device output provided with such a haptic system.

The present invention relates to a haptic system. The present invention also relates to a method for manufacturing a shape memory liquid crystal network. Furthermore, the present invention relates to electronic apparatuses with user input and device output provided with such a haptic system.

Haptic technology, also known as kinaesthetic communication or 3D touch, refers to any technology that can create an experience of touch by applying forces, vibrations, or motions to the user. These technologies can be used to create virtual objects in a computer simulation, to control virtual objects, and to enhance remote control of machines and devices. Haptic devices may incorporate tactile sensors that measure forces exerted by the user on the interface. Simple haptic devices are common in the form of game controllers, joysticks, and steering wheels.

US 2021/149489 relates to a touchpad apparatus comprising: a bottom layer comprising processing circuitry, a tactile pixel layer disposed on top of the bottom layer, the tactile pixel layer comprising a plurality of tactile pixels, wherein the processing circuitry is configured to control operation of the plurality of tactile pixels through application of one or more stimuli to each tactile pixel and each tactile pixel is independently operable, and a surface layer disposed on top of the tactile pixel layer. The surface layer comprises a deformable material, wherein each tactile pixel comprises a top plate comprising a plurality of vertices; and a support strut coupled to each vertex of the plurality of vertices, each support strut comprising a liquid crystal elastomer (LCE) hinge disposed between a first rigid portion and a second rigid portion. US 2022/069198 relates to a method of preparing a shape-reconfigurable micropatterned polymer thin film, the method comprising (a) forming a polymer alignment layer on each of two patterned electrode substrates; (b) fabricating a sandwich electrode cell by cross-assembling the two patterned electrode substrates on which the polymer alignment layer is formed at regular intervals; (c) injecting a liquid-crystalline organic monomer mixture between the two substrates of the sandwich electrode cell; and (d) producing a micropatterned polymer thin film by performing photocuring of the mixture in a state in which an electric field is applied to the sandwich electrode cells containing the liquid-crystalline organic monomer mixture therein. The liquid-crystalline organic monomer mixture contains a liquid-crystalline organic monomer having at least one acrylic group attached thereto and a photoinitiator, wherein the liquid-crystalline organic monomer is at least one selected from the group consisting of 4-(3-acryloyloxypropyloxy)-benzoic acid 2-methyl-1,4-phenylene ester, 4-methoxybenzoic acid 4-(6-acryloyloxy-hexyloxy)phenyl ester, 4-cyanophenyl-4′-(6-acryloyloxyhexyloxy)benzoate, and 1,4-bis-[4-(6-acryloyloxyhexyloxy)benzoyloxy]-2-methylbenzene. Step (d) comprises irradiating UV light at an intensity of 10 to 200 mW for 1 minute to 2 hours, wherein the polymer alignment layer in step (a) is formed by coating the patterned electrode substrate with a solution of molecular-phobic polyimide, followed by curing. The liquid crystal monomers form upon polymerization a polymer, and preferably a crosslinked polymer, resembling the initial molecular order of the of the liquid crystal monomer mixture. The achieved polymer or polymer network is further referred to as liquid crystal polymer, liquid crystal network or liquid crystal polymer network.

EP 3 136 224 relates to a haptic feedback generator, comprising a structural material, a bistable material configured in a first bistable configuration associated with the structural material, a first actuator coupled to the bistable material which when activated causes the bistable material to move from the first bistable configuration to a second bistable configuration, thereby generating haptic feedback, and a first actuator activation signal receiver, which upon receipt of an actuator activation signal, initiates activation of the first actuator, wherein the bistable material comprises carbon fibers embedded in a polymer matrix, or wherein the bistable material is a liquid crystal polymer.

US 2013/0154984 relates to a haptic system comprising a panel-type display device, an information selection haptic panel which is set on a top surface of said panel-type display device, a shape memory alloy which contracts upon electrification and heating to make said information selection haptic panel move, and an insulating heat conductor which disperses heat which was generated by said shape memory alloy. CN 110524861 relates to a processing method for preparing a shape memory product by using crystalline thermotropic shape memory polymers, including pure linear polymers, cross-linked polymers, and blends of linear polymers and cross-linked polymers.

EP 2 502 210 relates to a method for manufacturing a security label, comprising providing a layer comprising a shape memory polymer, heating of the shape memory polymer layer via a first switching temperature of the shape memory polymer, imprinting of a three-dimensional surface structure containing information for identifying the product in the shape memory polymer layer, and cooling of the shape memory polymer layer at a fixing temperature of the shape memory polymer to convert the shape memory polymer layer into the first state.

A number of publications provide local haptic information through static, not switchable, structures. Other publications relate haptic feedback by make the whole device vibrating or provide the haptic effects by local bending of a plastic film.

An object of the present invention is to develop programmable and locally deformable surfaces capable of large deformations in microscale.

Another object of the present invention is to apply haptic technology at a smaller scale, making the technology suitable for devices that value compactness and lightness.

Another object of the present invention is to integrate haptic surfaces into electric devices and enable untethered actuation.

The present invention thus relates to a haptic system comprising: a substrate provided with a surface layer,
the surface layer comprising a shape memory liquid crystal network which contracts upon electric and/or thermal stimuli to achieve a smoother surface or to form protrusions in the surface layer.

The present inventors found that pre-cured shape memory liquid crystal networks can be used to create programmable haptic surfaces. An important aspect of the present invention is the local molecular order retained in the polymer film and its response to stimuli that distorts this order.

In an example the shape memory liquid crystal network comprises a pre-cured LCN (liquid crystal polymer network).

In an example the shape memory crystal network is obtained by a method comprising two stages, wherein in a first stage a loosely crosslinked network is obtained by reaction between a liquid crystalline diacrylate and a dithiol in the presence of tri- or four functional thiol, and wherein in a second stage a deformation is established in the network that causes orientation of the obtained liquid crystal chains that are fixed by a photo crosslinking reaction.

In an example the shape memory crystal network is obtained by a method comprising two stages, wherein in a first stage a loosely crosslinked network is obtained by reaction between a liquid crystalline diacrylate and an amine, and wherein in a second stage a deformation is established in the network that causes orientation of the obtained liquid crystal chains that are fixed by a photo crosslinking reaction.

As mentioned, the liquid crystal network is made in two steps, corresponding to two states of the polymer between which it is switched. In a first step a surface profile is brought in a loosely crosslinked network obtained by a reaction between a liquid crystal monomer, referred to as a reactive mesogen and often consisting of a rodlike central chemical structure to which reactive groups, such as acrylate groups are attached at both sides or the molecular rod by a spacer group, such as an alkylene unit, and a difunctional compound, such as a dithiol or an amine, that can react with the functional group such that a polymer is formed of relative short chain length, further referred to as oligomer. The loose crosslinking comes from a polyfunctional crosslinker, such as a tri- of tetra-thiol, that is added in small quantity and co-reacts when the oligomer is formed. The reaction mixture is chosen such that at the ends of the oligomer one or more reactive groups are available for further reaction in a second step. This loosely crosslinked oligomer takes the surface of the mould against which it is polymerized. In a second step the surface of the loosely crosslinked polymer network is deformed by a second mould which changes the topography and stretches the loosely crosslinked oligomer and the oligomer chains become oriented. In this state the loosely crosslinked oligomer is crosslinked by a further reaction of the reactive end groups initiated by temperature or light by means of a dissolved initiator.

The properties of the polymer are such that its glass transition temperature is relatively low, at room temperature or below, and that the liquid crystal transition temperature to the isotropic phase is within the range of 30 to 120° C. The surface of the obtained polymer has the shape introduced by the second mould at room temperature and takes the shape given by the first mould at elevated temperature around the liquid crystal transition temperature to the isotropic phase, or higher. The switching between the two states is reversible.

The present invention also relates to a method for manufacturing a shape memory liquid crystal network, comprising the following:
reacting components on a substrate;
removing a solvent;
pressing the surface of the substrate in a desired shape thereby forming a local alignment within the molecules of the pressed area;
polymerizing the molecules of the pressed area for arresting the prior formed local alignments.

In an example the substrate is a flexible or rigid substrate, such as glass or plastic.

In an example the step of polymerizing is carried out with UV light.

The present invention also relates to an electronic apparatus with user input and device output provided with a haptic system as discussed above.

In an example the electronic apparatus is chosen from the group of smartphones, desktop monitors, displays, computer mousses, smart watches, and VR systems.

The present invention also relates to vehicles wherein steering wheels of cars are provided with a haptic system as discussed above for providing warnings to its user thereof.

The present invention also relates to surgical robots with control handles provided with a haptic system as discussed above for remotely controlling relaying information on pressure.

In an example the non-programmed material is formed by reacting all the dissolved components on a flexible or rigid substrate/surface, such as glass or plastic, and then removing the solvent. The surface is then programmed by pressing in the desired shape; for instance, pressing a circular shape in the material would cause the significant changes in topography once the material is activated. The strain caused by pressing in a shape onto the material forms a local alignment within the molecules of the pressed area. Then the programmed shape is secured by polymerizing the material with UV light, arresting prior formed local alignments. The material can then be activated thermally to either achieve a smoother surface or form the desired protrusions; the actuation occurs because the thermal stimuli disrupts the local alignments. Thus, once the thermal stimuli is removed and the material is allowed to cool again, the material returns to its original shape. The activated protrusions can be sensed by human fingers easily.

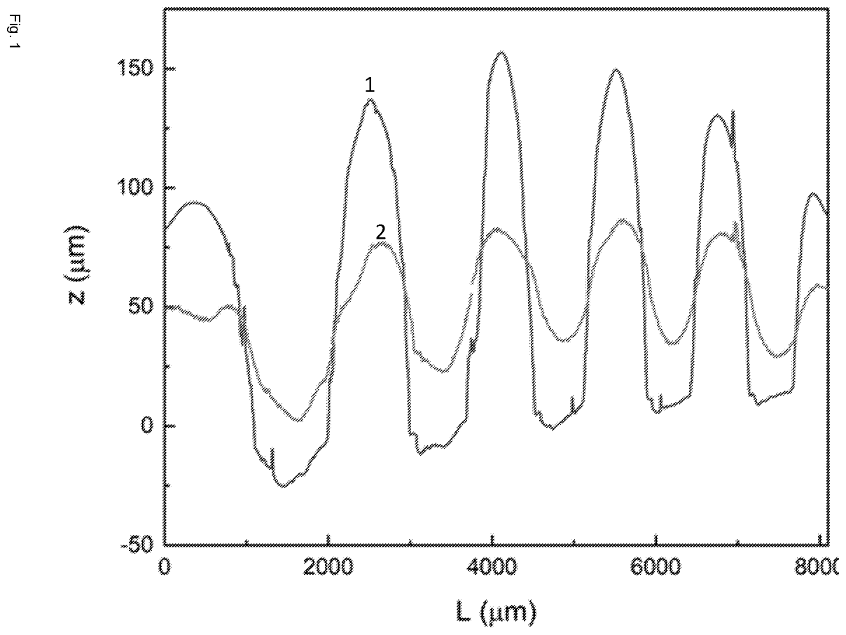

FIG. 1 shows a surface profile of the material upon activation and deactivation, i.e. a height profile of a coating on a glass substrate. The black curve gives the profile at room temperature after several heating and cooling cycles. The red line is showing the profile of the activated material at 120° C.

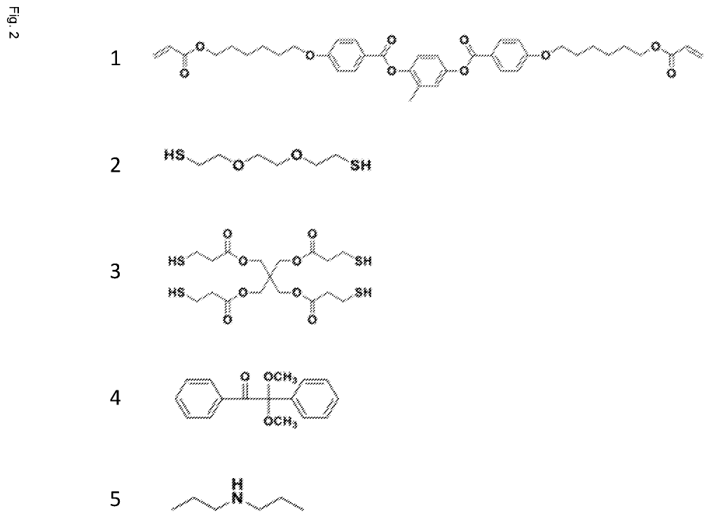

FIG. 2 shows the materials used for the fabrication of haptic surfaces.

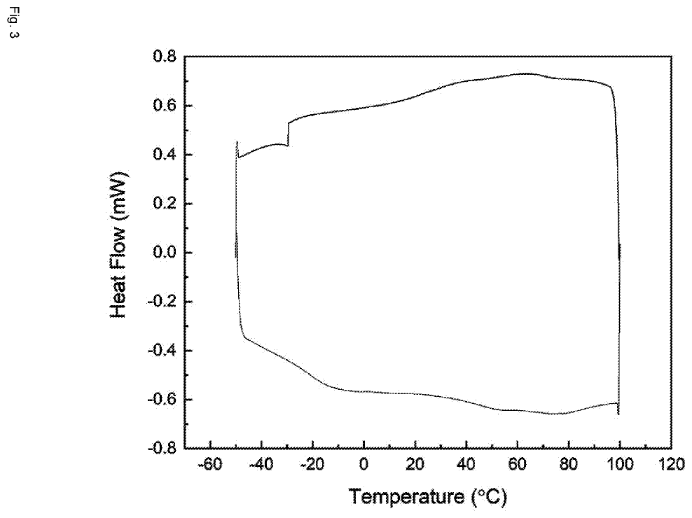

FIG. 3 shows a DSC profile of LC polymer.

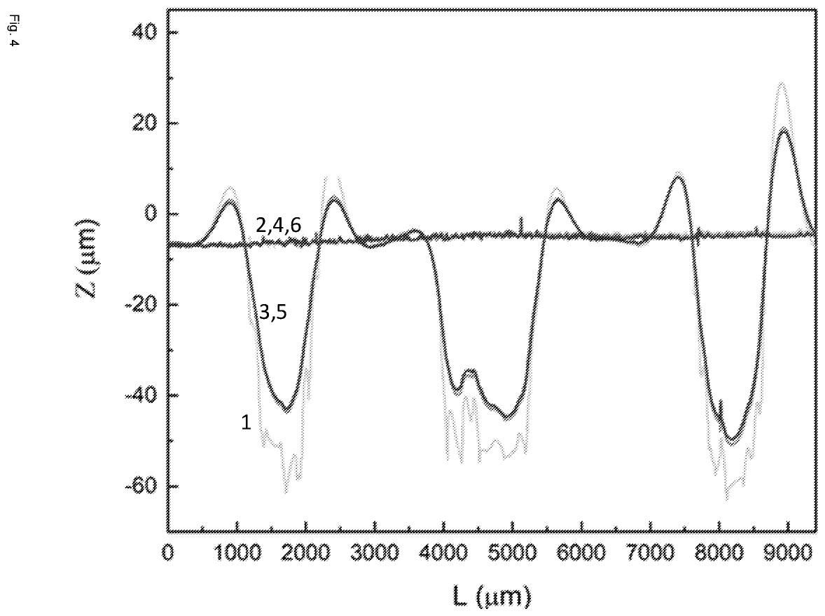

FIG. 4 shows a surface profile of LCE coating during activation and deactivation.

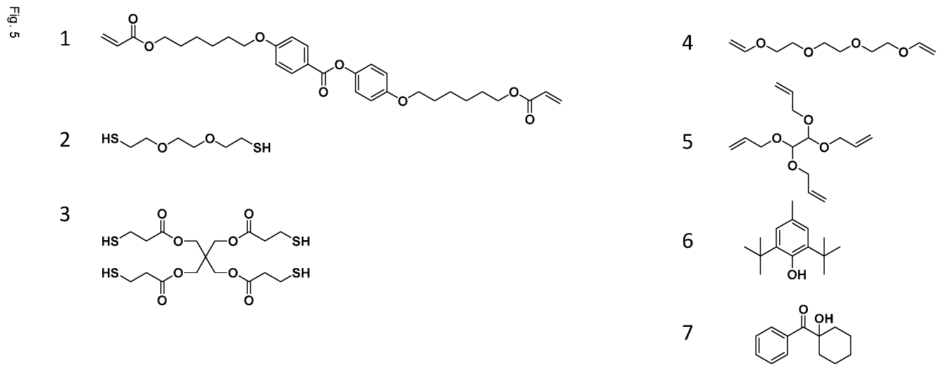

FIG. 5 shows the materials used for the fabrication of haptic surfaces.

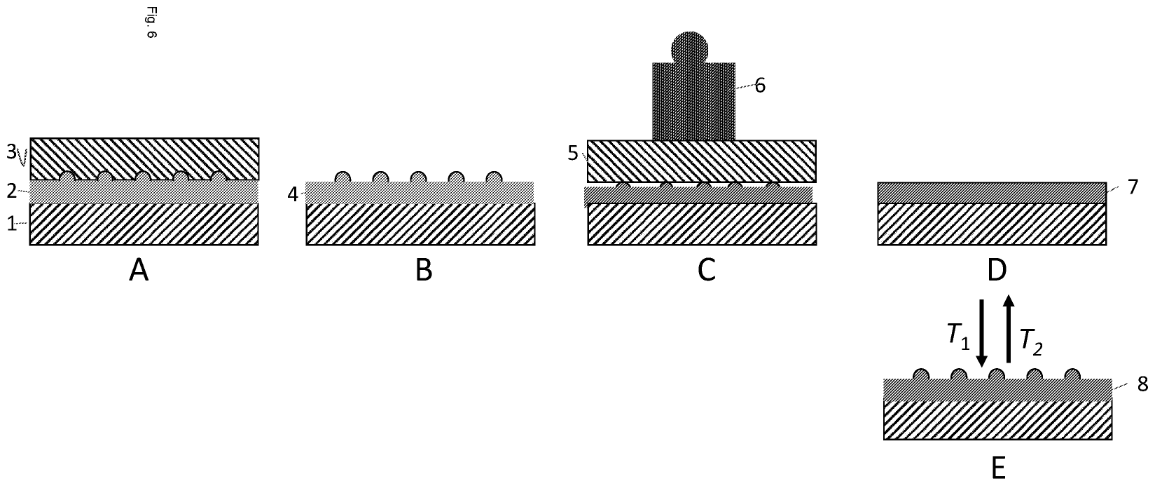

FIG. 6 shows the process steps for the fabrication of haptic surfaces that switches between flat in the non-activated state to corrugated in the activated state.

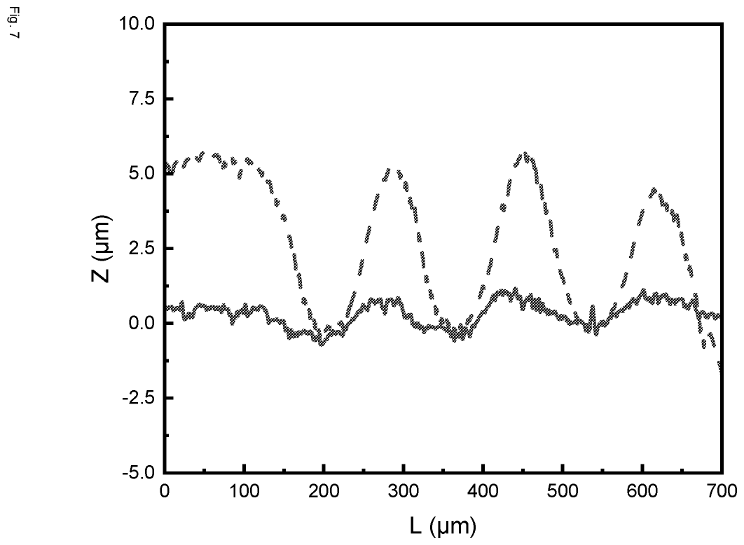

FIG. 7 shows a surface profile of a liquid crystal network coating.

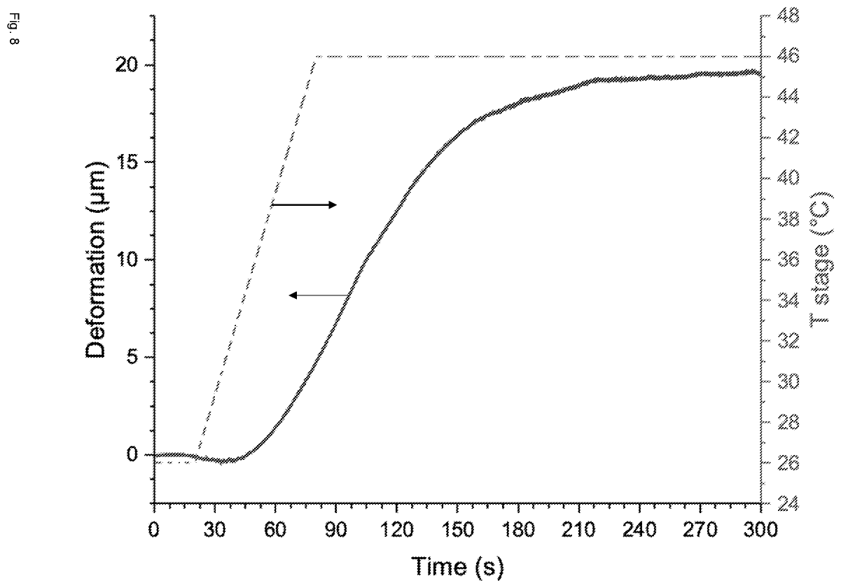

FIG. 8 shows the relation between time and deformation, together with a temperature profile used to actuate the surface.

The present invention will allow the application of haptics not only in larger, bulkier devices like game controllers, but also in devices that value compactness and lightness, such as smartphones. Using the present invention, the screen of the smartphone can be programmed to physically deform upon user input, which can be detected by the user's sense of touch; this would create a phone that feels you as much as you feel the phone.

The application of the present invention will not be limited to smartphones; all types of electronics with user input and device output, such as basic appliances like desktop monitors, could enhance their device output by integrating the present invention.

Moreover, devices with user input and device output, such as VR systems, can make use of the present invention. In fact, in a VR environment, haptics is incredibly important for user experience; being able to accurately experience touch feedback in a virtual world would be an incredible leap for the technology.

The present haptic surfaces can also be used in the wider world in applications such as the steering wheels of cars providing warnings, control handles of remotely controlled surgical robots relaying information on pressure, haptic displays for visually-impaired people, surface of an intelligent computer mouse, etc.

Hereinafter, the present invention will be described in detail.

In FIG. 2 the materials used for the fabrication of haptic surfaces are shown. Monomer 1 was purchased from Merck. Crosslinker 2 and catalyst 5 were purchased from Sigma Aldrich. Crosslinker 3 was purchased from Bruno Bock. Photoinitiator 4was purchased from Ciba Specialty Chemicals. Liquid crystal (LC) oligomer for haptic surfaces were fabricated by thiol Michael addition reaction from a mixture containing 75% of monomer 1, 19.6% of crosslinker 2, 4.3% of crosslinker 3 and 1.1% of photoinitiator 4 and catalytic amount of catalyst 5. In some experiments the concentration of crosslinker 2 was varied while the mutual ratio of other components were kept the same. The mixture was prepared by dissolving the components in dichloromethane. Catalyst 5 was added to the mixture last. FIG. 2 shows the materials used for the fabrication of haptic surfaces. Reagent 1 is a reactive liquid crystal monomer, 4-(3-acryloyloxyhexyloxy)-benzoic acid 2-methyl-1,4-phenylene ester. Reagent 2 is a dithiol chain extender, 3,6-dioxa-1,8-octanedithiol. Reagent 3 is the tetrafunctional thiol crosslinker pentaerythritol tetrakis (3-mercaptopropionate). Reagent 4 is the photoinitiator, commercially available under the name Irgacure 651. Reagent 5 is the catalyst promoting the reaction between the acrylate groups and thiol groups. The reaction ratios are chosen such that in the first reaction step there is an excess of acrylate groups. Consequently, the loosely crosslinked oligomers have acrylate end groups which will polymerize in the UV initiated reaction in the second stage.

FIG. 3 shows a differential scanning calorimetric (DSC) measurement of LC polymer. It shows a glass transition temperature of around −30° C. and a broad transition from the liquid crystalline to the isotropic phase at around 80° C.

FIG. 4 shows a surface profile of LCE coating during activation and deactivation. Curve 1 is the surface obtained directed after the process. The flat lines 2, 4 and 6 represent the flat surface profile during heating at 120° C. Curves 3 and 5 are the surface profile after cooling to room temperature after actuation step 2 and 4 respectively.

For preparing the sample the mixture was coated on clean glass substrates and left for drying overnight at room temperature. Desired dentures were made by placing 3D printed molds with programmed geometrical parameters onto the coating and applying pressure on top of the mold for 30 minutes. LC oligomer coatings were then photo crosslinked by UV exposure at 30° C. for 30 minutes under N2 using a mercury lamp.

Alternatively, the mixture was coated on a plastic substrate provided with a negative surface structure of surface elements that has to be brought in the sample surface, hereafter called the mould. After drying overnight, the mixture was transferred to a second substrate, provided with miniaturized resistive heating element after which the mould was removed. This gives a coated substrate with protrusions in the surface of the coating. Then in a second step the obtained coated substrate was pressed with a flat mould on its top surface such that the protrusions deform to become flat. During this process flow takes place that orient the liquid crystal molecules which will later be responsible for the switching behaviour. In this flat state, still under pressure, the sample is photo crosslinked and remain flat after removal of the flat mould. By local heating the protrusions are retained and removed again upon cooling.

Thermal analysis was carried out with differential scanning calorimetry (DSC). DSC analysis of the liquid crystal polymer is given in FIG. 3. The surface topography of the coatings was measured using a confocal microscopy (Sensofar). Confocal microscopic images show the topography of the dynamic surface before and after activation. Analysis of the profile showed an increase in the dentures around 40 μm on average which corresponds to 20% of the coating thickness, which can be observed in FIG. 4. Different textures were fabricated by using different 3D printed molds.

Confocal microscopic images show the topography of the dynamic surface prepared with a concentric mold before and after activation. Analysis of the profile showed an increase in the dentures around 25 μm on average which can be observed in FIG. 1.

In an embodiment of the present invention as discussed above, in a first step of the preparation of the coating a surface topographic structure is already brought in by polymerizing against a mould. In this way a flat surface is formed with bumps. This forms the later stable state two. For this reaction, the reaction mixture contains a reactive liquid crystal diacrylate monomer, a chain extender dithiol and a crosslinker tetrafunctional thiol which reacts together via a catalysed addition reaction. The molecular ratios are chosen such that after this oligomerization reaction the end groups of the oligomer chains are thiols. In the next step the film is brought to a flat state by press and photo crosslinked in this state. For this second reaction the reaction mixture contains a di-vinyl chain extender and a tetrafunctional vinyl crosslinker which react under UV light with the oligomer to form a more densely crosslinked, but still flexible, network. This reaction is initiated by a photoinitiator but can also be carried out thermally in the presence of a thermal free-radical initiator. After completion of this reaction stable state one is formed after which the mould can be removed. The surface of this second mould determines the topography of the surface that is stable at room temperature. The film can now reversibly switch between state one (flat) and by heating above the isotropic temperature of the polymer film to the corrugated state two. By cooling, the sample switches back to the flat state. The transition between flat and corrugated can be experienced by touch.

FIG. 5 shows the reagents used for this process in order to realize an actuation at relatively low temperatures. Reagent 1 is the rod-like reactive mesogen 4-(6-(acryloyloxy)hexyloxy)phenyl 4-(6-(acryloyloxy)hexyloxy)benzoate, used in the reaction mixture in a quantity of 61.5 w %. Reagent 2 is chain extender dithiol reacting with reagent 1: 3,6-dioxa-1,8-octanedithiol, used in the reaction mixture in a quantity of 16.3 w %. Reagent 3 is the polyfunctional thiol pentaerythritol tetrakis (3-mercaptopropionate), used in the reaction mixture in a quantity of 13.4 w %, This reagent is used to slightly crosslink the oligomer formed by reagents 1 and 2 in the first reaction step. Reagent 4 is a reactive difunctional alkyl ether: triethylene glycol divinyl ether, used in the reaction mixture in a quantity of 4.0 w %. Reagent 5 is the polyfunctional vinyl crosslinker glyoxal bis(diallyl acetal), used in the reaction mixture in a quantity of 0.3 w %. Together with reagent 4 it reacts under UV light at the second reaction step to set the final surface topography. Reagent 6 is inhibitor 2,6-di-tert-butyl-4-methylphenol, used in a concentration of 0.6 w %. Reagent 7 is the photoinitiator for the second reaction as is commercially available under the name Irgacure 184 and is added in a concentration of 1.9 w %.

FIG. 6 shows the process flow to fabricate a surface that switches between flat in the non-activated state to corrugated in the activated state. In step A substrate 1 is covered with the initial reaction mixture consisting of reagents 1 to 7 given in FIG. 5. Mould 3 is pressed on this structure prior to curing after which the reaction proceeds until full conversion of all acrylate groups. Then mould 3 is removed leaving a structured coating 4 on the substrate. Next a flat mould 5 is pressed on the loosely crosslinked coating by weight until the structures of the coating deform to become flat. In this stage the completion of the reaction takes place by polymerization of the vinyl and thiol end groups as shown in FIG. 5, reagents with reference numbers 2 to 5, initiated by UV light actuating reagent 7, see FIG. 5. Then mould 5 is removed to provide a close to flat coating 7 which is the final active layer. By raising the temperature to T₁ the coating surface deforms thereby giving coating 8. By cooling to temperature T₂, the coating deforms back to its initial state 7. Typically, temperature T₁ is the same or close to the nematic to isotropic transition of the liquid crystal network which in the case of the reagents given in FIG. 5 is around 40° C. The temperature T₂ is room temperature or close to that.

FIG. 7 shows a surface profile of a liquid crystal network coating, made from the materials shown in FIG. 5 and made following the procedure given in FIG. 6, switching between the deactivated state at room temperature and the activated state at 50° C. The solid line represents the non-activated state and dotted line the activated state by heating at 50° C.

FIG. 8 shows the switching speed from a flat surface to a corrugated surface (solid line) together with temperature profile (dotted line) used to actuate the surface.

It is part of the present invention that the heating to switch between the two states can be performed locally by integrated miniaturized heating elements, such that a pattern can be obtained that can be read by hand contact.

The present invention also relates to a surface of which the friction coefficient can be adjusted by the height of the protrusions.

The present invention also relates to a transparent coating provided with a surface that switches between a flat, optical clear state and a state that enhances the writing comfort of a stylus on display screen

The present invention also relates to a surface of which the aero-dynamic and fluid-dynamics properties can be adjusted by the height of the protrusions.

1. A haptic system having a programmable haptic surface layer comprising:
a substrate provided with a surface layer,
the surface layer comprising a shape memory crystal network which contracts upon electric and/or thermal stimuli to achieve a smoother surface or to form protrusions in the surface layer, wherein in a first step of the preparation of the shape memory crystal network a surface topographic structure is already brought in by polymerizing against a mould.

2. The haptic system according to claim 1, wherein the shape memory crystal network comprises a pre-cured LCN (liquid crystal polymer network).

3. The haptic system according to claim 1, wherein the shape memory crystal network is obtained by a method comprising two stages, wherein in a first stage a loosely crosslinked network is obtained by reaction between a liquid crystalline diacrylate and a dithiol in the presence of tri- or four functional thiol, and wherein in a second stage a deformation is established in the network that causes orientation of the obtained liquid crystal chains that are fixed by a photo crosslinking reaction.

4. The haptic system according to claim 1, wherein the shape memory crystal network is obtained by a method comprising two stages, wherein in a first stage a loosely crosslinked network is obtained by reaction between a liquid crystalline diacrylate and an amine, and wherein in a second stage a deformation is established in the network that causes orientation of the obtained liquid crystal chains that are fixed by a photo crosslinking reaction.

5. A method for manufacturing a shape memory liquid crystal network, comprising the following:
reacting components on a substrate;
removing a solvent;
pressing the surface of the substrate in a desired shape thereby forming a local alignment within the molecules of the pressed area;
polymerizing the molecules of the pressed area for arresting the prior formed local alignments.

6. The method according to claim 5, wherein the substrate is a flexible or rigid substrate, such as glass or plastic.

7. The method according to claim 5, wherein polymerizing is carried out with UV light.

8. The method according to claim 5, wherein the shape memory liquid crystal network is obtained in two stages, wherein in a first stage a loosely crosslinked network is obtained by reaction between a liquid crystalline diacrylate and a dithiol in the presence of tri- or four functional thiol, and wherein in a second stage a deformation is established in the network that causes orientation of the obtained liquid crystal chains that are fixed by a photo crosslinking reaction.

9. The method according to claim 5, wherein the shape memory liquid crystal network is obtained in two stages, wherein in a first stage a loosely crosslinked network is obtained by reaction between a liquid crystalline diacrylate and an amine, and wherein in a second stage a deformation is established in the network that causes orientation of the obtained liquid crystal chains that are fixed by a photo crosslinking reaction.

10. Electronic apparatus with user input and device output provided with a haptic system according to claim 1.

11. Electronic apparatus according to claim 10, wherein the apparatus is chosen from the group of smartphones, desktop monitors, displays, computer mousses, smart watches, and VR systems.

12. Vehicles wherein steering wheels of cars are provided with a haptic system according to claim 1 for providing warnings to its user thereof.

13. Surgical robots with control handles provided with a haptic system according to claim 1 for remotely controlling relaying information on pressure.

14. The use of the height of protrusions of a haptic system according to claim 1 for adjusting the friction coefficient of a surface layer.

15. The use of the height of protrusions of a haptic system according to claim 1 for adjusting the aero-dynamic and fluid-dynamics properties of a surface layer.

16. A transparent coating provided with a surface that switches between a flat, optical clear state and a state that enhances the writing comfort of a stylus on display screen.
